(19) United States
(12) Patent Application Publication
(10) Pub. No.: US 2010/0239515 A1
(43) Pub. Date: Sep. 23, 2010

(54) COSMETIC COMPOSITION CONTAINING A GLYCINE COMPOUND

(75) Inventor: Stephane ARDITTY, Ballainvilliers (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/723,713

(22) Filed: Mar. 15, 2010

Related U.S. Application Data
(60) Provisional application No. 61/161,823, filed on Mar. 20, 2009.

(30) Foreign Application Priority Data
Mar. 18, 2009 (FR) 09 51721

(51) Int. Cl. A61K 8/36; A61Q 1/10

(52) U.S. Cl. 424/70.7

(57) ABSTRACT

Composition useful for coating the eyelashes containing an aqueous phase and an emulsifying system including at least one glycine derivative and/or a salt thereof.

### REFERENCE TO PRIOR APPLICATIONS

This application claims priority to U.S. provisional application Ser. No. 61/161,823, filed Mar. 20, 2009; and to French patent application 09 51721, filed Mar. 18, 2009, both incorporated herein by reference.

### FIELD OF THE INVENTION

The present patent application relates to the field of cosmetics, particularly to the make-up or care of the eyelashes, and to mascaras, especially water-based mascaras, etc.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Compositions for coating the eyelashes, such as mascaras, are generally make-up compositions, compositions to be applied to a make-up (also known as top coat) or also compositions for the cosmetic care of the eyelashes.

Mascaras are prepared in particular according to two types of formulation: water-based mascaras, known as cream mascaras, in the form of a dispersion of waxes in water; anhydrous mascaras or mascaras with a low water content, known as waterproof mascaras, in the form of dispersions of waxes in organic solvents.

The application of mascara makes it possible to increase the volume of the eyelashes and consequently to increase the intensity of the gaze. There exist numerous thickening or volumizing mascaras for doing this, the principle of which consists in depositing as much material as possible on the eyelashes so as to obtain a volumizing (or charging) effect.

It is in particular through the amount of solid particles (in particular waxes, which make it possible to structure the composition) that the specific features of use desired for the compositions can be adjusted, such as, for example, their fluidity or consistency, and also their thickening power (also known as charging or make-up power).

These solid particles are dispersed in the cream mascara using a surfactant system.

The conventional emulsifiers or emulsifying systems include in particular emulsifying systems based on triethanolamine stearate.

Application FR 2 908 298 also describes mascaras comprising, as emulsifying system, glycine derivatives of acylglycinate type of formula (I) where R is an acyl group:

R—HNCH₂COOX (I)

or glycinates of following formula (II), with R₁ being a hydrocarbon chain comprising from 10 to 30 carbon atoms and R₂ being a primary alcohol comprising from 2 to 10 carbon atoms:

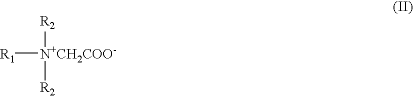

However, compositions for making up the eyelashes obtained with these emulsifying systems exhibit a relatively fluid texture, which requires a minimum amount of waxes (i.e., of the order of at least 20% by weight) in order to obtain an a fortiori charging mascara texture.

### DETAILED DESCRIPTION OF THE PREFERRED EMBODIMENTS

One problem solved by the present patent application is that of providing a mascara in which not only the waxes but also the pigments are homogeneously dispersed, the mascara exhibiting a texture which is sufficiently thick to obtain a volumizing charging deposited layer on the eyelashes and exhibiting a satisfactory consistency which makes possible ready application to the eyelashes and a smooth and homogeneous deposited layer.

Preferably, the mascara according to the invention additionally comprises a minimum amount of waxes, which makes it possible to obtain a charging mascara, while retaining a degree of latitude for incorporating other starting materials capable of being of advantage in regard to the performances of hold or of cosmetic quality, for example.

The inventors of the present patent application have, surprisingly and unexpectedly, solved this problem using an emulsifying system comprising at least one glycine compound (derivative) of the amphoacetate type of following formula (A) and/or one of its salts:

R—CO—NH(CH₂)₂N(CH₂CH₂OH)CH₂COO⁻X⁺ (A)

where R is a carbon chain comprising at least 14 carbon atoms.

The inventors of the present patent application were able to observe that the emulsifying system defined in the present patent application makes possible good dispersion of the pigments and/or waxes; this dispersion is of the quality of those obtained with emulsifying systems based on triethanolamine stearate. This composition makes it possible to obtain a charging make-up for the eyelashes and a satisfactory consistency compatible with producing a smooth and homogeneous deposited layer on the fibres, this being the case in particular with a smaller amount of waxes than a composition based on acylglycinate or on glycinate of the formula (II).

The compositions in accordance with the invention can have a viscoelastic behaviour.

Generally, a material is said to be viscoelastic when, under the effect of shearing, it has both the characteristics of an elastic material, that is to say capable of storing energy, and the characteristics of a viscous material, that is to say capable of dissipating energy.

The viscoelastic behaviour of the compositions in accordance with the invention can be more particularly characterized by its rigidity modulus G. This parameter is defined in particular in the work “Initiation à la rhéologie” [Introduction to Rheology], G. Couarraze and J. L. Grossiord, 2^{nd} edition, 1991, published by Lavoisier-Tec 1 Doc.

The measurements are carried out on an RS 600 controlled-stress rheometer from ThermoRheo equipped with a thermostatically controlled bath and with a stainless steel rotor of cone/plate geometry, with a diameter of 35 mm and an angle of 2°. The two surfaces are “sandblasted” to limit phenomena of sliding at the walls.

The measurements are carried out at 25° C.±1° C.

The dynamic measurements are carried out while applying a harmonic variation of the stress. In these experiments, the amplitudes of the shear stress (recorded as τ) and of the shear strain (recorded as γ) are low, so as to remain within the limits of the linear viscoelastic region of the composition (conditions which make it possible to evaluate the rheological characteristics of the composition at rest).

The linear viscoelastic region is generally defined by the fact that the response of the material (i.e. the strain) is at any moment directly proportional to the value of the force applied (i.e. the stress). In this region, the stresses applied are low and the material undergoes strains without modifying its microscopic structure. Under these conditions, the material is studied “at rest” and nondestructively.

The composition is subjected to harmonic shearing according to a stress τ(t) varying sinusoidally according to an angular frequency ω (ω=2Πν), ν being the frequency of the shearing applied. The composition thus sheared is subjected to a stress τ(t) and responds according to a strain γ(t) corresponding to microstrains for which the rigidity modulus varies little as a function of the stress imposed.

The stress τ(t) and the strain γ(t) are defined respectively by the following relationships:

τ(t)=τ₀ cos(ω·t) γ(t)=γ₀ cos(ω·t−δ)

τ₀ being the maximum amplitude of the stress and γ₀ being the maximum amplitude of the strain. The elasticity δ is the phase angle between the stress and the strain.

The measurements are carried out at a frequency of 1 Hz (ν=1 Hz).

Increasing stresses are applied to the sample, starting from an initial stress equal to 0.01 Pa, to arrive at a final stress of 1000 Pa, the stresses only being applied once.

The change in the rigidity modulus G (corresponding to the ratio of τ₀ to γ₀) and in the elasticity δ (corresponding to the phase angle of the stress applied with respect to the strain measured) is thus measured as a function of the stress τ(t) applied.

The strain of the composition for the stress zone in which the variation in the rigidity modulus G is less than 7% (microstrain zone) is measured in particular and thus the “plateau” parameter Gp is determined.

The composition exhibits, for example, a plateau rigidity modulus Gp of greater than or equal to 10 Pa, preferably greater than or equal to 50 Pa, which can range up to 10⁶ Pa and better still up to 5×10⁵ Pa.

A first subject-matter of the present patent application is a composition useful for example in coating the eyelashes comprising an aqueous phase and an emulsifying system comprising at least one glycine derivative of amphoacetate type of following formula (A) and/or a cosmetically acceptable salt of the derivative:

R—CO—NH(CH₂)₂N(CH₂CH₂OH)CH₂CH₂COO⁻X⁺ (A)

where R is a carbon chain comprising at least 14 carbon atoms.

A second subject-matter of the present patent application is a method for making up or for the nontherapeutic care of the eyelashes comprising the application, to the eyelashes, of the composition according to the present patent application.

A third subject-matter of the present patent application is the uses of the composition according to the present patent application, in particular the use of this composition for obtaining a homogeneous and/or volumizing make-up of the eyelashes.

Other characteristics, properties and advantages of the present invention will become more clearly apparent on reading the description and examples which follow.

### Emulsifying System

The emulsifying system of the composition according to the invention comprises at least one glycine derivative of the amphoacetate type of the following formula (A) and/or a cosmetically acceptable salt of the said the derivative:

R—CO—NH(CH₂)₂N(CH₂CH₂OH)CH₂COO⁻X⁺ (A)

where R is a carbon chain comprising at least 14 carbon atoms, and
X represents a cation chosen, for example, from ions of alkali metals, such as sodium, lithium or potassium, potassium, preferably sodium or potassium, from ions of alkaline earth metals, such as magnesium, ammonium groups and their mixtures.

The glycine derivative or its salt is preferably chosen from the salts of following formula (A):

R—CO—NH(CH₂)₂N(CH₂CH₂OH)CH₂COO⁻X⁺ (A)

in which R represents a linear or branched and saturated or unsaturated hydrocarbon chain comprising at least 14 carbon atoms, preferably from 14 to 30 carbon atoms, preferably from 14 to 24 carbon atoms and better still from 14 to 22 carbon atoms, and X represents a cation chosen, for example, from ions of alkali metals such as Na, Li or K, preferably Na or K, from ions of alkaline earth metals, such as Mg, ammonium groups and their mixtures.

The R—CO group can be chosen in particular from fatty acids derived from palm oil, fatty acids derived from olive oil, fatty acids derived from sweet almond oil, fatty acids derived from babassu oil, fatty acids derived from cocoa oil and their mixtures.

Preferably, R—CO is a fatty acid derived from palm oil.

Mention may be made, as compound of formula (A), for example of the compounds carrying the INCI names sodium palmamphoacetate, which compound is present in particular in the commercial mixture Resassol AGP from Res Pharma, or sodium olivamphoacetate, such as, for example, Resassol AGO from Res Pharma, or sodium sweet-almondamphoacetate, such as, for example, Resassol AGM from Res Pharma, or sodium babassuamphoacetate, such as, for example, Resassol AGB from Res Pharma, or sodium cacaoamphoacetate, such as, for example, Resassol AGC from Res Pharma.

The glycine derivatives of formula (A) and their salts can be present in the composition in a content ranging for example from 0.1 to 20% by weight, preferably from 0.5 to 15% by weight and better still from 1 to 10% by weight, with respect to the total weight of the composition. According to a particular embodiment, the glycine derivatives of formula (A) and their salts ranges for example from 2 to 10% by weight, and still precisely from 4 to 8% by weight, with respect to the total weight of the composition.

Preferably, the glycine derivative or derivatives of formula (A) and their salts constitute the main surfactant system of the composition.

The term “main surfactant system” is understood to mean a system which, in its absence, does not result in the formation of a stable composition.

The term “stable” is understood to mean a composition which, after having been placed in an oven at 45° C. for two months, does not exhibit, after returning to ambient temperature, grains perceptible to the touch when a fine layer of the composition is sheared between the fingers.

Advantageously, the glycine derivative(s) of formula (A) and their salts constitute the sole surfactant system of the composition.

The term “sole” is understood to mean that any optional additional surfactant system is present in a content not exceeding 1% and preferably not exceeding 0.5%. More preferably, the term “sole” denotes the complete absence of any other surfactant system.

The composition according to the invention comprises, of course, a physiologically acceptable medium. The term “physiologically acceptable compound or medium” is understood to mean, within the meaning of the present patent application, a compound or medium, the use of which is compatible with application to the eyelashes.

### Aqueous Phase

The composition according to the invention comprises an aqueous phase which can form the continuous phase of the composition.

The term “composition comprising an aqueous continuous phase” is understood to mean that the composition exhibits a conductivity, measured at 25° C., of greater than or equal to 23 μS/cm (microSiemens/cm), the conductivity being measured, for example, using an MPC227 conductivity meter from Mettler Toledo and an Inlab730 conductivity measurement cell. The measurement cell is immersed in the composition, so as to remove the air bubbles liable to be formed between the two electrodes of the cell. The conductivity is read as soon as the value of the conductivity meter has stabilized. A mean is determined over at least 3 successive measurements.

The aqueous phase comprises water and/or at least one water-soluble solvent.

The term “water-soluble solvent” denotes, in the present invention, a compound which is liquid at ambient temperature and which is miscible with water (miscibility in water of greater than 50% by weight at 25° C. and atmospheric pressure).

The water-soluble solvents which can be used in the compositions according to the invention can in addition be volatile.

Mention may in particular be made, among the water-soluble solvents which can be used in the compositions in accordance with the invention, of lower monoalcohols having from 1 to 5 carbon atoms, such as ethanol and isopropanol, or glycols having from 2 to 8 carbon atoms, such as ethylene glycol, propylene glycol, 1,3-butylene glycol and dipropylene glycol.

The aqueous phase (water and optionally the water-miscible solvent) is generally present in the composition according to the present patent application in a content ranging for example from 1% to 95% by weight, with respect to the total weight of the composition, preferably ranging for example from 3% to 80% by weight and preferentially ranging for example from 5% to 60% by weight. According to a particular embodiment, the aqueous phase is in a content ranging for example from 35% to 65% by weight and still better ranging for example from 40% to 60%.

The emulsifying system can moreover comprise at least one additional surface-active agent appropriately chosen in order to obtain a wax-in-water or oil-in-water emulsion.

Use may in particular be made of an emulsifier having, at 25° C., an HLB balance (hydrophilic-lipophilic balance), within the Griffin meaning, of greater than or equal to 8.

These additional surface-active agents can be chosen from nonionic, anionic, cationic or amphoteric surface-active agents or surface-active emulsifiers. Reference may be made to the document “Encyclopedia of Chemical Technology, Kirk-Othmer”, volume 22, pp. 333-432, 3^{rd} edition, 1979, Wiley, for the definition of the properties and functions (emulsifying) of surfactants, in particular pp. 347-377 of this reference for the anionic, amphoteric and nonionic surfactants.

These additional surfactants can preferably be chosen from:
a) nonionic surface-active agents with an HLB of greater than or equal to 8 at 25° C., used alone or as a mixture; mention may in particular be made of:
   oxyethylenated and/or oxypropylenated ethers (which can comprise from 1 to 150 oxyethylene and/or oxypropylene groups) of glycerol;
   oxyethylenated and/or oxypropylenated ethers (which can comprise from 1 to 150 oxyethylene and/or oxypropylene groups) of fatty alcohols (in particular of C₈-C₂₄ and preferably C₁₂-C₁₈ alcohols), such as the oxyethylenated ether of cetearyl alcohol comprising 30 oxyethylene groups (CTFA name “Ceteareth-30”), the oxyethylenated ether of stearyl alcohol comprising 20 oxyethylene groups (CTFA name “Steareth-20”), such as the Brij 78 sold by Uniqema, and the oxyethylenated ether of the mixture of C₁₂-C₁₅ fatty alcohols comprising 7 oxyethylene groups (CTFA name “C12-15 Pareth-7”), sold under the name Neodol 25-7® by Shell Chemicals,
   esters of fatty acid (in particular of C₈-C₂₄ and preferably C₁₆-C₂₂ acid) and of polyethylene glycol (which can comprise from 1 to 150 ethylene glycol units), such as PEG-50 stearate and PEG-40 monostearate, sold under the name Myrj 52P® by ICI Uniquema,
   esters of fatty acid (in particular of C₈-C₂₄ and preferably C₁₆-C₂₂ acid) and of oxyethylenated and/or oxypropylenated glycerol ethers (which can comprise from 1 to 150 oxyethylene and/or oxypropylene groups), such as PEG-200 glyceryl monostearate, sold under the name Simulsol 220 TM® by Seppic; polyethoxylated glyceryl stearate comprising 30 ethylene oxide groups, such as the product Tagat S® sold by Goldschmidt, polyethoxylated glyceryl oleate comprising 30 ethylene oxide groups, such as the product Tagat O® sold by Goldschmidt, polyethoxylated glyceryl cocoate comprising 30 ethylene oxide groups, such as the product Varionic L® 13® sold by Sherex, polyethoxylated glyceryl isostearate comprising 30 ethylene oxide groups, such as the product Tagat L® sold by Goldschmidt, and polyethoxylated glyceryl laurate comprising 30 ethylene oxide groups, such as the product Tagat I® from Goldschmidt,
   esters of fatty acid (in particular of C₈-C₂₄ and preferably C₁₆-C₂₂ acid) and of oxyethylenated and/or oxypropylenated sorbitol ethers (which can comprise from 1 to 150 oxyethylene and/or oxypropylene groups), such as polysorbate 20, sold under the name Tween 20® by Croda, or polysorbate 60, sold under the name Tween 60® by Uniqema,
   dimethicone copolyol, such as that sold under the name Q2-5220® by Dow Corning,
   dimethicone copolyol benzoate (Finsolv SLB 101® and 201® from Fintex),
   copolymers of propylene oxide and of ethylene oxide, also known as EO/PO polycondensates,
   and their mixtures.

The EO/PO polycondensates are more particularly copolymers consisting of polyethylene glycol and polypropylene glycol blocks, such as, for example, polyethylene glycol/polypropylene glycol/polyethylene glycol triblock polycondensates. These triblock polycondensates have, for example, the following chemical structure:

H—(O—CH₂—CH₂)ₐ—(O—CH(CH₃)—CH₂)_{b}—(O—CH₂—CH₂)ₐ—OH

in which formula a ranges for example from 2 to 120 and b ranges for example from 1 to 100.

The EO/PO polycondensate preferably has a weight-average molecular weight ranging for example from 1000 to 15 000 and better still ranging form 2000 to 13 000. Advantageously, the EO/PO polycondensate has a cloud point, at 10 g/l in distilled water, of greater than or equal to 20° C., preferably of greater than or equal to 60° C. The cloud point is measured according to Standard ISO 1065. Mention may be made, as EO/PO polycondensate which can be used according to the invention, of the polyethylene glycol/polypropylene glycol/polyethylene glycol triblock polycondensates sold under the Synperonic® names, such as Synperonic PE/L44® and Synperonic PE/F127®, by ICI.
b) nonionic surface-active agents with an HLB of less than 8 at 25° C., optionally in combination with one or more nonionic surface-active agents with an HLB of greater than 8 at 25° C., such as mentioned above, such as:
   esters and ethers of monosaccharides, such as sucrose stearate, sucrose cocoate, sorbitan stearate and their mixtures, such as Arlatone 2121®, sold by ICI;
   oxyethylenated and/or oxypropylenated ethers (which can comprise from 1 to 150 oxyethylene and/or oxypropylene groups), of fatty alcohols (in particular of C₈-C₂₄ and preferably C₁₂-C₁₈ alcohols), such as the oxyethylenated ether of stearyl alcohol comprising 2 oxyethylene groups (CTFA name “Steareth-2”);
   esters of fatty acids (in particular of C₈-C₂₄ and preferably C₁₆-C₂₂ acid) and of polyol, in particular of glycerol or of sorbitol, such as glyceryl stearate, such as the product sold under the name Tegin M® by Goldschmidt, glyceryl laurate, such as the product sold under the name Imwitor 312® by Hüls, polyglyceryl-2 stearate, sorbitan tristearate or glyceryl ricinoleate;
   lecithins, such as soya lecithins (for example Emulmetik 100 J from Cargill or Biophilic H from Lucas Meyer);
   the cyclomethicone/dimethicone copolyol mixture sold under the name Q2-3225C® by Dow Corning.
c)/anionic surfactants, such as:
   salts of C₁₆-C₃₀ fatty acids, in particular those deriving from amines, such as triethanolamine stearate and/or 2-amino-2-methylpropane-1,3-diol stearate; however, preferably, the composition according to the present patent application comprises less than 1% of triethanolamine stearate;
   salts of polyoxyethylenated fatty acids, in particular those deriving from amines or the alkali metal salts, and their mixtures;
   phosphoric esters and their salts, such as “DEA oleth-10 phosphate” (Crodafos N 10N from Croda) or monocetyl monopotassium phosphate (Amphisol K from Givaudan);
   sulfosuccinates, such as “Disodium PEG-5 citrate lauryl sulfosuccinate” and “Disodium ricinoleamido MEA sulfosuccinate”;
   alkyl ether sulphates, such as sodium lauryl ether sulphate;
   isethionates;
   acylglutamates, such as “Disodium hydrogenated tallow glutamate” (Amisoft HS-21 R®, sold by Ajinomoto) and sodium stearoyl glutamate (Amisoft HS-11 PF® sold by Ajinomoto), and their mixtures;
   soya derivatives, such as potassium soyate;
   citrates, such as glyceryl stearate citrate (Axol C 62 Pellets from Degussa);
   proline derivatives, such as sodium palmitoyl proline (Sepicalm VG from Seppic) or the mixture of sodium palmitoyl sarcosinate, magnesium palmitoyl glutamate, palmitic acid and palmitoyl proline (Sepifeel One from Seppic);
   lactylates, such as sodium stearoyl lactylate (Akoline SL from Karlshamns AB);
   sarcosinates, such as sodium palmitoyl sarcosinate (Nikkol sarcosinate PN) or the 75/25 mixture of stearoyl sarcosine and myristoyl sarcosine (Crodasin SM from Croda);
   sulphonates, such as sodium C14-17 alkyl sec sulphonate (Hostapur SAS 60 from Clariant);
   acylglycinates of formula (I) and glycinates of formula (II) above, such as sodium cocoyl glycinate (Amilite GCS-12 from Ajinomoto);
   and their mixtures.

The composition according to the invention can also comprise, in addition to the glycine derivative of formula (A), one or more amphoteric surfactants, such as N-acylamino acids, for example N-alkylaminoacetates and disodium cocoamphodiacetate, and amine oxides, such as stearamine oxide, or also silicone surfactants, such as dimethicone copolyol phosphates, for example that sold under the name Pecosil PS 100® by Phoenix Chemical.

In the composition in accordance with the invention, the total content of additional surface-active agents can range from 1 to 3% by weight, with respect to the total weight of the composition, preferably from 1 to 20% by weight and better still from 2 to 15% by weight.

According to one alternative form, the composition according to the present patent application comprises less than 1% by weight, preferably less than 0.5% by weight, of triethanolamine and better still is devoid of triethanolamine.

According to a preferred alternative form, the cosmetic composition according to the present patent application comprises less than 1% by weight, preferably less than 0.5% by weight, of triethanolamine stearate and better still is devoid of triethanolamine stearate.

According to one embodiment, the composition additionally comprises a cosurfactant chosen from fatty alcohols, preferably comprising from 10 to 30 carbon atoms. The term “fatty alcohol comprising from 10 to 30 carbon atoms” is understood to mean any pure, saturated or unsaturated, branched or unbranched, fatty alcohol comprising from 10 to 30 carbon atoms.

Use is preferably made of a fatty alcohol comprising from 10 to 26 carbon atoms, better still from 10 to 24 carbon atoms and even better still from 14 to 22 carbon atoms.

Mention may in particular be made, as fatty alcohols which can be used in the composition, of lauryl, myristyl, cetyl, stearyl, oleyl, cetearyl (mixture of cetyl alcohol and stearyl alcohol), behenyl or erucyl alcohols and their mixtures. Use is preferably made of cetyl alcohol.

Such fatty alcohols are sold in particular under the name Nafol by Sasol.

The fatty alcohol can be present in a content ranging for example from 0.2 to 20% by weight, preferably from 0.3 to 10% by weight, with respect to the total weight of the composition.

### Wax(es)

The composition according to the present patent application advantageously comprises at least one wax.

The term “wax” is understood to mean, within the meaning of the present invention, a lipophilic compound which is solid at ambient temperature (25° C.), which exhibits a reversible solid/liquid change in state and which has a melting point of greater than or equal to 30° C. which can range up to 120° C.

The melting point of the wax can be measured using a differential scanning calorimeter (DSC), for example the calorimeter sold under the name DSC 30 by Metier.

The waxes can be hydrocarbon, fluorinated and/or silicone waxes and can be of vegetable, mineral, animal and/or synthetic origin. In particular, the waxes exhibit a melting point of greater than 25° C. and better still of greater than 45° C.

The wax can be present in a content ranging for example from 0.1 to 35% by weight, with respect to the total weight of the composition, better still from 1 to 30% by weight and even better still from 5 to 25% by weight. In a particular embodiment, the wax is present in a content ranging for example from 15 to 35% by weight, with respect to the total weight of the composition.

Use may in particular be made of hydrocarbon waxes, such as beeswax, lanolin wax and Chinese insect waxes; rice wax, carnauba wax, candelilla wax, ouricury wax, esparto wax, cork fibre wax, sugarcane wax, Japan wax and sumac wax; montan wax, microcrystalline waxes, paraffin waxes and ozokerite; polyethylene waxes, waxes obtained by the Fischer-Tropsch synthesis and waxy copolymers, and also their esters.

Mention may also be made of the waxes obtained by catalytic hydrogenation of animal or vegetable oils having linear or branched C₈-C₃₂ fatty chains.

Mention may in particular be made, among these, of hydrogenated jojoba oil, hydrogenated sunflower oil, hydrogenated castor oil, hydrogenated coconut oil, hydrogenated lanolin oil, di(1,1,1-trimethylolpropane) tetrastearate, sold under the name “Hest 2T-4S” by Heterene, or di(1,1,1-trimethylolpropane) tetrabehenate, sold under the name “Hest 2T-4B” by Heterene.

Mention may also be made of silicone waxes, such as alkyl or alkoxy dimethicones having from 16 to 45 carbon atoms, or fluorinated waxes.

Use may also be made of the wax obtained by hydrogenation of olive oil esterified with stearyl alcohol sold under the name “Phytowax Olive 18 L 57” or else of the waxes obtained by hydrogenation of castor oil esterified with cetyl alcohol sold under the names “Phytowax castor 16L64” and “Phytowax castor 22L73” by Sophim. Such waxes are described in Application FR-A-2 792 190.

According to a specific embodiment, the compositions in accordance with the invention can comprise at least one wax known as “tacky wax”, that is to say having a tack of greater than or equal to 0.7 N·s and a hardness of less than or equal to 3.5 MPa.

The use of a tacky wax can in particular make it possible to obtain a cosmetic composition which is easily applied to the eyelashes, which has good attachment to the eyelashes and which results in the formation of a smooth, homogeneous and thickening make-up.

The tacky wax used can in particular have a tack ranging for example from 0.7 N·s to 30 N·s, in particular of greater than or equal to 1 N·s, in particular ranging for example from 1 N·s to 20 N·s, especially of greater than or equal to 2 N·s, in particular ranging for example from 2 N·s to 10 N·s, and especially ranging for example from 2 N·s to 5 N·s.

The tack of the wax is determined by the measurement of the change in the force (compressive force or stretching force) as a function of the time at 20° C. using the texture analyser sold under the name “TA-TX2i®” by Rheo, equipped with a spindle made of acrylic polymer in the shape of a cone forming an angle of 45°.

The measurement protocol is as follows:

The wax is melted at a temperature equal to the melting point of the wax+10° C. The molten wax is cast in a receptacle with a diameter of 25 mm and a depth of 20 mm. The wax is recrystallized at ambient temperature (25° C.) for 24 hours, so that the surface of the wax is flat and smooth, and then the wax is stored at 20° C. for at least 1 hour before measuring the tack.

The spindle of the texture analyser is displaced at the rate of 0.5 mm/s and then penetrates the wax to a penetration depth of 2 mm. When the spindle has penetrated the wax to a depth of 2 mm, the spindle is held stationary for 1 second (corresponding to the relaxation time) and is then withdrawn at the rate of 0.5 mm/s.

During the relaxation time, the force (compressive force) strongly decreases until it becomes zero and then, during the withdrawal of the spindle, the force (stretching force) becomes negative to subsequently again increase towards the value of 0. The tack corresponds to the integral of the curve of the force as a function of the time for the part of the curve corresponding to the negative values of the force (stretching force). The value of the tack is expressed in N·s.

The tacky wax which can be used generally has a hardness of less than or equal to 3.5 MPa, in particular ranging for example from 0.01 MPa to 3.5 MPa, especially ranging for example from 0.05 MPa to 3 MPa, indeed even ranging for example from 0.1 MPa to 2.5 MPa.

The hardness is measured according to the protocol described above.

Use may be made, as tacky wax, of a C₂₀-C₄₀ alkyl (hydroxystearyloxy)stearate (the alkyl group comprising from 20 to 40 carbon atoms), alone or as a mixture, in particular a C₂₀-C₄₀ alkyl 12-(12′-hydroxystearyloxy)stearate, of formula (II):

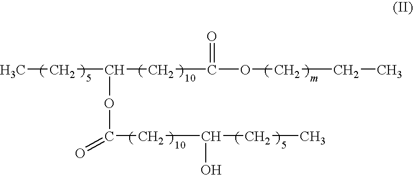

in which m is an integer ranging for example from 18 to 38, or a mixture of compounds of formula (II).

Such a wax is sold in particular under the names “Kester Wax K 82 P®” and “Kester Wax K 80 P®” by Koster Keunen.

The abovementioned waxes generally exhibit a starting melting point of less than 45° C.

Use may also be made of the microcrystalline wax sold under the reference SP18 by Strahl and Pitsch, which exhibits a hardness of approximately 0.46 MPa and a tack value of approximately 1 N·s.

The wax or waxes can be present in the form of an aqueous wax microdispersion. The term “aqueous wax microdispersion” is understood to mean an aqueous dispersion of wax particles in which the size of the wax particles is less than or equal to approximately 1 μm.

Wax microdispersions are stable dispersions of colloidal wax particles and are described in particular in “Microemulsions Theory and Practice”, edited by L. M. Prince, Academic Press (1977), pages 21-32.

In particular, these wax microdispersions can be obtained by melting the wax in the presence of a surfactant and optionally of a portion of the water and then gradually adding hot water with stirring. The intermediate formation of an emulsion of the water-in-oil type, followed by phase inversion, with a microemulsion of oil-in-water type finally being obtained, is observed. On cooling, a stable microdispersion of solid colloidal wax particles is obtained.

The wax microdispersions can also be obtained by stirring the mixture of wax, of surfactant and of water using stirring means, such as ultrasound, a high pressure homogenizer or turbine mixers.

The particles of the wax microdispersion preferably have mean sizes of less than 1 μm (in particular ranging for example from 0.02 μm to 0.99 μm), preferably of less than 0.5 μm (in particular ranging for example from 0.06 μm to 0.5 μm).

These particles are composed essentially of a wax or of a mixture of waxes. However, they can comprise a minor proportion of oily and/or pasty fatty additives, a surfactant and/or a conventional fat-soluble additive/active principle.

The compositions according to the present patent application can also comprise at least one pasty compound.

“Pasty” is understood to mean, within the meaning of the present invention, a lipophilic fatty compound having a reversible solid/liquid change in state and comprising, at the temperature of 23° C., a liquid fraction and a solid fraction.

The pasty compound preferably has a hardness at 20° C. ranging for example from 0.001 to 0.5 MPa, preferably from 0.002 to 0.4 MPa.

The hardness is measured according to a method of penetration of a probe into a sample of compound and in particular using a texture analyser (for example, the TA-XT21 from Stable Micro Systems Ltd) equipped with a stainless steel cylinder with a diameter of 2 mm. The hardness measurement is carried out at 20° C. at the centre of 5 samples. The cylinder is introduced into each sample at a pre-rate of 1 mm/s and then at a measuring rate of 0.1 mm/s, the depth of penetration being 0.3 mm. The value recorded for the hardness is that of the maximum peak.

This pasty compound is in addition, at the temperature of 23° C., in the form of a liquid fraction and of a solid fraction. In other words, the starting melting point of the pasty compound is less than 23° C. The liquid fraction of the pasty compound measured at 23° C. represents from 23 to 97% by weight of the compound. This liquid fraction at 23° C. preferably represents between 40 and 85% by weight of the compound.

The liquid fraction by weight of the pasty compound at 23° C. is equal to the ratio of the enthalpy of fusion consumed at 23° C. to the enthalpy of fusion of the pasty compound.

The enthalpy of fusion of the pasty compound is the enthalpy consumed by the compound to change from the solid state to the liquid state. The pasty compound is “in the solid state” when the whole of its mass is in the solid form. The pasty compound is “in the liquid state” when the whole of its mass is in the liquid form.

The enthalpy of fusion of the pasty compound is equal to the area under the curve of the thermogram obtained using a differential scanning calorimeter (DSC), such as the calorimeter sold under the name MDSC 2920 by TA Instrument, with a rise in temperature of 5 or 10° C. per minute, according to Standard ISO 11357-3:1999. The enthalpy of fusion of the pasty compound is the amount of energy necessary to change the compound from the solid state to the liquid state. It is expressed in J/g.

The enthalpy of fusion consumed at 23° C. is the amount of energy absorbed by the sample to change from the solid state to the state which it exhibits at 23° C., composed of a liquid fraction and of a solid fraction.

The liquid fraction of the pasty compound measured at 32° C. preferably represents from 40 to 100% by weight of the compound, preferably from 50 to 100% by weight, preferably from 80 to 100% by weight, more preferably from 90 to 100% by weight of the compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the enthalpy of fusion consumed at 32° C. to the enthalpy of fusion of the pasty compound. The enthalpy of fusion consumed at 32° C. is calculated in the same way as the enthalpy of fusion consumed at 23° C.

The pasty compound is preferably chosen from synthetic compounds and compounds of vegetable origin. A pasty compound can be obtained by synthesis from starting materials of vegetable origin.

The pasty compound is advantageously chosen from:

lanolin and its derivatives, such as lanolin alcohol, oxyethylenated lanolins, acetylated lanolin, lanolin esters, such as isopropyl lanolate, or oxypropylenated lanolins,

polymeric or nonpolymeric silicone compounds, such as polydimethylsiloxanes with high molecular weights or polydimethylsiloxanes comprising side chains of the alkyl or alkoxy type having from 8 to 24 carbon atoms, in particular stearyl dimethicones,

polymeric or nonpolymeric fluorinated compounds,

vinyl polymers, in particular:
homopolymers of olefins
copolymers of olefins
homopolymers and copolymers of hydrogenated dienes
linear or branched oligomers which are homo- or copolymers of alkyl (meth)acrylates preferably having a C₈-C₃₀ alkyl group
oligomers which are homo- and copolymers of vinyl esters having C₈-C₃₀ alkyl groups
oligomers which are homo- and copolymers of vinyl ethers having C₈-C₃₀ alkyl groups,

fat-soluble polyethers resulting from the polyetherification between one or more C₂-C₁₀₀, preferably C₂-C₅₀, diols,

polyol ethers chosen from ethers of pentaerythritol and of polyalkylene glycol, ethers of fatty alcohol and of sugar, and their mixtures, the ether of pentaerythritol and of polyethylene glycol comprising 5 oxyethylene (50E) units (CTFA name: PEG-5 Pentaerythrityl Ether), the ether of pentaerythritol and of polypropylene glycol comprising 5 oxypropylene (5 OP) units (CTFA name: PPG-5 Pentaerythrityl Ether), and their mixtures, and more especially the PEG-5 Pentaerythrityl Ether, PPG-5 Pentaerythrityl Ether and soybean oil mixture sold under the name “Lanolide” by Vevy, in which the constituents occur in a 46/46/8 ratio by weight: 46% of PEG-5 Pentaerythrityl Ether, 46% of PPG-5 Pentaerythrityl Ether and 8% of soybean oil,

esters and polyesters,

and their mixtures.

The pasty compound is preferably a polymer, in particular a hydrocarbon polymer.

A preferred silicone and fluorinated pasty compound is polymethyl trifluoropropyl methylalkyl dimethyl-siloxane, manufactured under the name X22-1088 by Shin Etsu.

When the pasty compound is a silicone and/or fluorinated polymer, the composition advantageously comprises a compatibilizing agent, such as short-chain esters, for example isodecyl neopentanoate.

Preference is given, among fat-soluble polyethers, in particular to copolymers of ethylene oxide and/or of propylene oxide with long-chain C₆-C₃₀ alkylene oxides, more preferably such that the ratio by weight of the ethylene oxide and/or of the propylene oxide to alkylene oxides in the copolymer is from 5:95 to 70:30. In this family, mention will in particular be made of the copolymers such that the long-chain alkylene oxides are positioned in blocks having an average molecular weight of 1000 to 10 000, for example a polyoxyethylene/polydodecyl glycol block copolymer, such as the ethers of dodecanediol (22 mol) and of polyethylene glycol (450E) sold under the trade name Elfacos ST9 by Akzo Nobel.

Preference is given in particular, among the esters, to

esters of an oligomeric glycerol, in particular esters of diglycerol, especially condensates of adipic acid and of diglycerol, for which a portion of the hydroxyl groups of the glycerols has reacted with a mixture of fatty acids, such as stearic acid, capric acid, isostearic acid and 12-hydroxystearic acid, such as in particular those sold under the trade name Softisan 649 by Sasol

phytosterol esters,

pentaerythritol esters,

esters formed from
at least one alcohol, at least one of the alcohols being a Guerbet alcohol, and
a dimer diacid formed from at least one unsaturated fatty acid,
such as the ester of tall oil fatty acid dimer comprising 36 carbon atoms and of a mixture i) of Guerbet alcohols comprising 32 carbon atoms and ii) of behenyl alcohol, or the ester of linoleic acid dimer and of a mixture of two Guerbet alcohols, 2-tetra-decyloctadecanol (32 carbon atoms) and 2-hexadecylicosanol (36 carbon atoms),

noncrosslinked polyesters resulting from the polycondensation between a linear or branched C₄-C₅₀ dicarboxylic acid or polycarboxylic acid and a C₂-C₅₀ diol or polyol,

polyesters which result from the esterification, by a polycarboxylic acid, of an aliphatic hydroxycarboxylic acid ester, such as Risocast DA-L and Risocast DA-H, sold by the Japanese company Kokyu Alcohol Kogyo, which are esters resulting from the esterification reaction of hydrogenated castor oil with dilinoleic acid or isostearic acid,

ester aliphatic esters resulting from the esterification of an aliphatic hydroxycarboxylic acid ester by an aliphatic carboxylic acid (Salacos HCIS (V)-L, sold by Nishing Oil). The aliphatic carboxylic acid comprises from 4 to 30 and preferably from 8 to 30 carbon atoms. It is preferably chosen from hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, eicosanoic acid, isoarachidic acid, octyldodecanoic acid, heneicosanoic acid, docosanoic acid, and mixtures thereof. The aliphatic carboxylic acid is preferably branched. The aliphatic hydroxycarboxylic acid ester advantageously results from a hydroxylated aliphatic carboxylic acid comprising from 2 to 40 carbon atoms, preferably from 10 to 34 carbon atoms and better still from 12 to 28 carbon atoms and from 1 to 20 hydroxyl groups, preferably from 1 to 10 hydroxyl groups and better still from 1 to 6 hydroxyl groups. The aliphatic hydroxycarboxylic acid ester is chosen from:

a) partial or complete esters of saturated linear monohydroxylated aliphatic monocarboxylic acids;

b) partial or complete esters of unsaturated monohydroxylated aliphatic monocarboxylic acids;

c) partial or complete esters of saturated monohydroxylated aliphatic polycarboxylic acids;

d) partial or complete esters of saturated polyhydroxylated aliphatic polycarboxylic acids;

e) partial or complete esters of aliphatic C₂ to C₁₆ polyols which have reacted with a mono- or polyhydroxylated aliphatic mono- or polycarboxylic acid,

and their mixtures.

The ester aliphatic esters are advantageously chosen from:

the ester resulting from the esterification reaction of hydrogenated castor oil with isostearic acid in the proportions of 1 to 1 (1/1) or hydrogenated castor oil monoisostearate,

the ester resulting from the esterification reaction of hydrogenated castor oil with isostearic acid in the proportions of 1 to 2 (1/2) or hydrogenated castor oil diisostearate,

the ester resulting from the esterification reaction of hydrogenated castor oil with isostearic acid in the proportions of 1 to 3 (1/3) or hydrogenated castor oil triisostearate,

and their mixtures.

The pasty compound can also be of vegetable origin. In this case, mention may in particular be made of isomerized jojoba oil, such as the trans-isomerized partially hydrogenated jojoba oil manufactured or sold by Desert Whale under the commercial reference Iso-Jojoba-50®, orange wax, such as, for example, that which is sold under the reference Orange Peel Wax by Koster Keunen, shea butter, partially hydrogenated olive oil, such as, for example, the compound sold under the reference Beurrolive by Soliance, cocoa butter or mango oil, such as, for example, Lipex 302 from AarhusKarlshamn.

The pasty compounds can be present in the composition according to the invention in an amount of between 0.1 and 20% by weight, with respect to the total weight of the composition, preferably between 0.5 and 15% by weight and preferably between 1 and 10% by weight.

The compositions according to the present patent application can also comprise at least one hydrophilic or lipophilic film-forming polymer.

In the present patent application, the term “film-forming polymer” is understood to mean a polymer capable of forming, by itself alone or in the presence of an additional agent which is able to form a film, a macroscopically continuous film which adheres to the eyelashes and preferably a cohesive film and better still a film, the cohesion and the mechanical properties of which are such that the film can be isolable and handleable in isolation, for example when the film is produced by casting on a nonstick surface, such as a Teflon- or silicone-treated surface.

Generally, the content of “film-forming polymer” of the compositions according to the present patent application ranges for example from 0.1 to 40% by weight, preferably from 0.5 to 30% by weight and better still from 1 to 20% by weight, with respect to the total weight of the composition.

The hydrophilic film-forming polymer can be a water-soluble polymer or can be provided in dispersion in an aqueous medium.

Mention may be made, among the film-forming polymers which can be used in the composition of the present invention, of synthetic polymers of radical type or of polycondensate type, polymers of natural origin, and their mixtures.

Mention may be made, as examples of water-soluble film-forming polymers, of:

proteins, such as proteins of plant origin, such as wheat or soya protein; proteins of animal origin, such as keratins, for example keratin hydrolysates and sulphonic keratins;
cellulose polymers, such as hydroxyethylcellulose, hydroxypropylcellulose, methylcellulose, ethylhydroxy-ethylcellulose, carboxymethylcellulose and quaternized cellulose derivatives;

acrylic polymers or copolymers, such as polyacrylates or polymethacrylates;

vinyl polymers, such as polyvinylpyrrolidones, copolymers of methyl vinyl ether and of malic anhydride, the copolymer of vinyl acetate and of crotonic acid, copolymers of vinylpyrrolidone and of vinyl acetate, copolymers of vinylpyrrolidone and of caprolactam, or polyvinyl alcohol;

anionic, cationic, amphoteric or nonionic chitin or chitosan polymers;

gums arabic, guar gum, xanthan derivatives or karaya gum;

alginates and carrageenans;

glycoaminoglycans, hyaluronic acid and its derivatives;

shellac resin, gum sandarac, dammars, elemis or copals;

deoxyribonucleic acid;

mucopolysaccharides, such as chondroitin sulphates;

and their mixtures.

The film-forming polymer can also be present in the composition in the form of particles in dispersion in an aqueous phase, generally known under the name of latex or pseudolatex. The techniques for preparing these dispersions are well known to a person skilled in the art.

Use may be made, as aqueous film-forming polymer dispersion, of acrylic dispersions, sold under the names Neocryl XK-90®, Neocryl A-1070®, Neocryl A-1090®, Neocryl BT-62®, Neocryl A-1079® and Neocryl A-523® by Avecia-Neoresins, Dow Latex 432® by Dow Chemical, Daitosol 5000 AD® or Daitosol 5000 SJ® by Daito Kasey Kogyo; Syntran 5760® by Interpolymer or Allianz Opt® by Röhm & Haas, or also aqueous dispersions of polyurethane, sold under the names Neorez R-981® and Neorez R-974® by Avecia-Neoresins, Avalure UR-405®, Avalure UR-410®, Avalure UR-425®, Avalure UR-450®, Sancure 875®, Avalure UR-445® and Sancure 2060® by Noveon, Impranil 85® by Bayer or Aquamere H-1511® by Hydromer; sulphopolyesters, sold under the trade name Eastman AQ® by Eastman Chemical Products, vinyl dispersions, such as Mexomer PAM®, aqueous dispersions of poly(vinyl acetate), such as “Vinybran®” from Nisshin Chemical or those sold by Union Carbide, aqueous dispersions of terpolymer of vinylpyrrolidone, dimethylaminopropylmethacrylamide and lauryldimethylmethacrylamidopropylammonium chloride, such as Styleze W from ISP, aqueous dispersions of polyurethane/polyacrylic hybrid polymers, such as those sold under the references “Hybridur®” by Air Products or “Duromer®” by National Starch, dispersions of core/shell type: for example those sold by Atofina under the Kynar reference (core: fluorinated-shell: acrylic) or also those described in the document U.S. Pat. No. 5,188,899 (core: silica-shell: silicone), and their mixtures.

The lipophilic polymer can be in solution or in dispersion in a nonaqueous solvent phase.

The compositions according to the present patent application can also comprise at least one hydrophilic gelling agent; they can be chosen from:

homo- or copolymers of acrylic acid or methacrylic acid or their salts and their esters and in particular the products sold under the names Versicol F® or Versicol K® by Allied Colloid, Ultrahold 8® by Ciba-Geigy, polyacrylic acids of Synthalen K type,

copolymers of acrylic acid and of acrylamide, sold in the form of their sodium salt under the Reten® names by Hercules, sodium salts of polyhydroxycarboxylic acids, sold under the name Hydagen F® by Henkel,

copolymers of polyacrylic acids and of alkyl acrylates of Pemulen type,

AMPS (polyacrylamidomethylpropanesulphonic acid partially neutralized with aqueous ammonia and highly crosslinked), sold by Clariant,

AMPS/acrylamide copolymers of Sepigel® or Simulgel® type, sold by Seppic,

copolymers of AMPS and of alkyl methacrylates which are polyoxyethylenated (crosslinked or noncrosslinked),

associative polyurethanes, such as the polymer C₁₆-OE₁₂₀-C₁₆ from Servo Delden (sold under the name SER AD FX1100, molecule comprising a urethane functional group and with a weight-average molecular weight of 1300), OE being an oxyethylene unit, Rheolate 205 comprising a urea functional group, sold by Rheox, or also Rheolate 208 or 204 (these polymers being sold in the pure form), or DW 1206B from Röhm & Haas comprising a C₂₀ alkyl chain and comprising a urethane bond, sold at 20% of active material in water. Use may also be made of solutions or dispersions of these associative polyurethanes, in particular in water or in an aqueous/alcoholic medium. Mention may be made, by way of example of such polymers, of SER AD fx1010, SER AD FX1035 and SER AD 1070 from Servo Delden and Rheolate 255, Rheolate 278 and Rheolate 244, sold by Rheox. Use may also be made of the products DW 1206F and DW 1206J, and also Acrysol RM 184 or Acrysol 44, from Röhm & Haas, or alternatively Borchigel LW 44 from Borchers,

and their mixtures.

Some water-soluble film-forming polymers mentioned above can also act as water-soluble gelling agent.

The hydrophilic gelling agents can be present in the compositions according to the invention in a content ranging for example from 0.05 to 40% by weight, with respect to the total weight of the composition, preferably from 0.1 to 20% by weight and better still from 5 to 15% by weight.

The compositions according to the present patent application can also comprise at least one or more oils or organic solvents.

The term “oil or organic solvent” is understood to mean a nonaqueous body which is liquid at ambient temperature and atmospheric pressure. The oil can be volatile or nonvolatile.

The term “volatile oil or organic solvent” is understood to mean, within the meaning of the invention, any nonaqueous medium capable of evaporating on contact with keratinous substances in less than one hour at ambient temperature and atmospheric pressure. The volatile organic solvent or solvents and the volatile oils of the invention are volatile cosmetic organic solvents and oils which are liquid at ambient temperature and which have a non-zero vapour pressure, at ambient temperature and atmospheric pressure, ranging for example from 0.13 Pa to 40 000 Pa (10⁻³ to 300 mmHg), in particular ranging for example from 1.3 Pa to 13 000 Pa (0.01 to 100 mmHg) and more particularly ranging for example from 1.3 Pa to 1300 Pa (0.01 to 10 mmHg). The term “nonvolatile oil” is understood to mean an oil which remains on keratinous substances at ambient temperature and atmospheric pressure for at least several hours and which has in particular a vapour pressure of less than 10⁻³ mmHg (0.13 Pa).

The oil can be present in the composition in a content ranging for example from 0.05 to 30% by weight, preferably 0.1 to 15% by weight, with respect to the total weight of the composition. The composition according to the invention can comprise volatile oils and/or nonvolatile oils, and their mixtures.

The volatile oils (or organic solvents) can be hydrocarbon oils, silicone oils, fluorinated oils or their mixtures.

The term “hydrocarbon oil” is understood to mean an oil comprising mainly hydrogen and carbon atoms and optionally oxygen, nitrogen, sulphur or phosphorus atoms. Volatile hydrocarbon oils can be chosen from hydrocarbon oils having from 8 to 16 carbon atoms, in particular branched C₈-C₁₆ alkanes, such as C₈-C₁₆ isoalkanes of petroleum origin (also known as isoparaffins), such as isododecane (also known as 2,2,4,4,6-pentamethylheptane), isodecane or isohexadecane, for example the oils sold under the “Isopars®” or “Permethyls®” trade names, branched C₈-C₁₆ esters, isohexyl neopentanoate, and their mixtures. Other volatile hydrocarbon oils, such as petroleum distillates, in particular those sold under the “Shell Solt®” name by Shell, can also be used.

Use may also be made, as volatile oils, of volatile silicones, such as, for example, volatile linear or cyclic silicone oils, in particular those having a viscosity ≦6 centistokes (6×10⁻⁶ m²/s) and having in particular from 3 to 6 silicon atoms, these silicones optionally comprising one or more alkyl or alkoxy groups having 1 or 2 carbon atoms. Mention may in particular be made, as volatile silicone oil which can be used in the invention, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and their mixtures.

Use may also be made of volatile organic solvents, in particular fluorinated organic solvents, such as nonafluoromethoxybutane or perfluoromethylcyclopentane.

Each of the compositions in accordance with the invention can also comprise at least one nonvolatile oil or organic solvent which can be chosen in particular from nonvolatile hydrocarbon oils and/or silicone oils and/or fluorinated oils.

Mention may in particular be made, as nonvolatile hydrocarbon oil, of:

hydrocarbon oils of vegetable origin, such as triglycerides composed of esters of fatty acids and of glycerol, the fatty acids of which can have varied chain lengths from C₄ to C₂₄, it being possible for these chains to be linear or branched and saturated or unsaturated; these oils are in particular wheat germ, sunflower, grape seed, sesame, maize, apricot kernel, castor, shea, avocado, olive, soybean, sweet almond, palm, rapeseed, cottonseed, hazelnut, macadamia, jojoba, alfalfa, poppy, pumpkinseed, cucumber, blackcurrant seed, evening primrose, millet, barley, quinoa, rye, safflower, candlenut, passionflower or musk rose oil; or triglycerides of caprylic/capric acids, such as those sold by Stéarineries Dubois or those sold under the names Miglyol 810®, 812® and 818® by Dynamit Nobel,

synthetic ethers having from 10 to 40 carbon atoms;

linear or branched hydrocarbons of mineral or synthetic origin, such as liquid petrolatum, polydecenes, hydrogenated polyisobutene, such as Parleam oil, squalane, and their mixtures;

synthetic esters, such as the oils of formula R₁COOR₂ in which R₁ represents the residue of a linear or branched fatty acid comprising from 1 to 40 carbon atoms and R₂ represents a hydrocarbon chain, in particular a branched hydrocarbon chain, comprising from 1 to 40 carbon atoms, provided that R₁+R₂ is ≧10, such as, for example, Purcellin oil (cetearyl octanoate), isopropyl myristate, isopropyl palmitate, C₁₂ to C₁₅ alkyl benzoate, hexyl laurate, diisopropyl adipate, isononyl isononanoate, 2-ethylhexyl palmitate, isostearyl isostearate, octanoates, decanoates or ricinoleates of alcohols or of polyalcohols, such as propylene glycol dioctanoate; hydroxylated esters, such as isostearyl lactate or diisostearyl malate; and pentaerythritol esters;

fatty alcohols which are liquid at ambient temperature comprising a branched and/or unsaturated carbon chain having from 12 to 26 carbon atoms, such as octyldodecanol, isostearyl alcohol, oleyl alcohol, 2-hexyldecanol, 2-butyloctanol or 2-undecylpentadecanol;

higher fatty acids, such as oleic acid, linoleic acid or linolenic acid;

and their mixtures.

The nonvolatile silicone oils which can be used in one or other of the compositions (i) or (ii) in accordance with the invention can be polydimethylsiloxanes (PDMSs) which are nonvolatile, polydimethylsiloxanes comprising pendent alkyl or alkoxy groups and/or alkyl or alkoxy groups at the end of the silicone chain, groups each having from 2 to 24 carbon atoms, phenylated silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

The fluorinated oils which can be used in the compositions in accordance with the invention are in particular fluorosilicone oils, fluorinated polyethers or fluorinated silicones, such as described in the document EP-A-847 752.

The content of nonvolatile oil or organic solvent in the composition in accordance with the invention ranges for example from 0.01 to 30% by weight, in particular from 0.1 to 25% by weight and better still from 0.1 to 20% by weight, with respect to the total weight of the composition.

The compositions in accordance with the invention can also comprise at least one colouring material, such as pulverulent materials, fat-soluble dyes or water-soluble dyes.

The pulverulent colouring materials can be chosen from pigments and pearlescent agents.

The pigments can be white or coloured, inorganic and/or organic and coated or uncoated. Mention may be made, among inorganic pigments, of titanium dioxide, optionally treated at the surface, zirconium, zinc or cerium oxides, and also iron or chromium oxides, manganese violet, ultramarine blue, chromium hydrate and ferric blue. Mention may be made, among organic pigments, of carbon black, pigments of D & C type, and lakes, based on cochineal carmine, of barium, strontium, calcium or aluminium.

The pearlescent agents can be chosen from white pearlescent pigments, such as mica covered with titanium oxide or with bismuth oxychloride, coloured pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

The fat-soluble dyes are, for example, Sudan red, D&C Red 17, D&C Green 6, β-carotene, soybean oil, Sudan brown, D&C Yellow 11, D&C Violet 2, D&C Orange 5, quinoline yellow or annatto.

These colouring materials can be present in a content ranging for example from 0.01 to 30% by weight, with respect to the total weight of the composition.

The compositions in accordance with the invention can also comprise at least one filler.

The fillers can be chosen from those well known to a person skilled in the art and commonly used in cosmetic compositions. The fillers can be inorganic or organic and lamellar or spherical. Mention may be made of talc, mica, silica, kaolin, powders formed of polyamide, such as Nylon®, sold under the name Orgasol® by Atochem, of poly-β-alanine and of polyethylene, powders formed of tetrafluoroethylene polymers, such as Teflon®, lauroyllysine, starch, boron nitride, hollow polymer microspheres which are expanded, such as those of poly(vinylidene chloride)/acrylonitrile, for example those sold under the name Expancel® by Nobel Industrie, acrylic powders, such as those sold under the name Polytrap® by Dow Corning, particles formed of polymethyl methacrylate and silicone resin microbeads (Tospearls® from Toshiba, for example), precipitated calcium carbonate, magnesium carbonate, basic magnesium carbonate, hydroxyapatite, hollow silica microspheres (Silica Beads® from Maprecos), glass or ceramic microcapsules, or metal soaps derived from organic carboxylic acids having from 8 to 22 carbon atoms and in particular from 12 to 18 carbon atoms, for example zinc stearate, magnesium stearate, lithium stearate, zinc laurate or magnesium myristate.

Use may also be made of a compound capable of swelling when heated and in particular of heat-expandable particles, such as nonexpanded microspheres formed of vinylidene chloride/acrylonitrile/methyl methacrylate copolymer or of copolymer of homopolymer of acrylonitrile, such as, for example, those sold respectively under the references Expancel® 820 DU 40 and Expancel® 007WU by Akzo Nobel.

The fillers can represent from 0.1 to 25% by weight, in particular from 0.2 to 20% by weight, with respect to the total weight of the composition.

The compositions in accordance with the invention can also comprise at least one fibre which makes possible an improvement in the lengthening effect.

The term “fibre” should be understood as meaning an object with a length L and a diameter D such that L is much greater than D, D being the diameter of the circle in which the cross section of the fibre is framed. In particular, the L/D ratio (or aspect ratio) is chosen within the range from 3.5 to 2500, in particular from 5 to 500 and more particularly from 5 to 150.

The fibres which can be used in the composition of the invention can be fibres of synthetic or natural and inorganic or organic origin. They can be short or long, individual or organized, for example plaited, and hollow or solid. They can have any shape and can in particular be circular or polygonal (square, hexagonal or octagonal) in cross section, according to the specific application envisaged. In particular, their ends are blunted and/or polished to prevent injury.

In particular, the fibres have a length ranging for example from 1 μm to 10 mm, in particular from 0.1 mm to 5 mm and more particularly from 0.3 mm to 3.5 mm. Their cross section can be included within a circle with a diameter ranging for example from 2 nm to 500 μm, in particular ranging for example from 100 nm to 100 μm and more particularly ranging for example from 1 μm to 50 μm. The weight or count of the fibres is often given in denier or decitex and represents the weight in grams per 9 km of yarn. The fibres according to the invention can in particular have a count chosen within the range from 0.15 to 30 denier and in particular from 0.18 to 18 denier.

The fibres which can be used in the composition of the invention can be chosen from rigid or nonrigid fibres. They can be of synthetic or natural and inorganic or organic origin.

Furthermore, the fibres may or may not be surface treated, may or may not be coated and may or may not be coloured.

Mention may be made, as fibres which can be used in the composition according to the invention, of fibres which are not rigid, such as polyamide) (Nylon° fibres, or fibres which are rigid, such as polyimideamide fibres, for example those sold under the Kermel° or Kermel Tech® names by Rhodia, or poly(p-phenylene terephthalamide) (or aramid) fibres, sold in particular under the Kevlar® name by DuPont de Nemours.

The fibres can be present in the composition according to the invention in a content ranging for example from 0.01% to 10% by weight, with respect to the total weight of the composition, in particular from 0.1% to 5% by weight and more particularly from 0.3% to 3% by weight.

The compositions in accordance with the invention can also comprise at least one cosmetic active principle.

Mention may in particular be made, as cosmetic active principles which can be used in the compositions in accordance with the invention, of antioxidants, preservatives, fragrances, neutralizing agents, emollients, thickeners, coalescence agents, plasticizers, moisturizing agents, vitamins and screening agents, in particular sunscreens, and their mixtures.

Of course, a person skilled in the art will take care to choose the optional additional additives and/or their amounts so that the advantageous properties of the composition according to the invention are not, or not substantially, detrimentally affected by the envisaged addition.

In a particular embodiment, the composition according to the invention has a dry matter content strictly lesser than to 450, preferably lesser than or equal to 42%, even better lesser than or equal to 400.

### The Dry Matter or Dry Extract Content is Measured as Follows.

The dry matter content, that is to say the content of nonvolatile material, may be measured in different ways; mention may be made for example of the methods based on drying in an oven, the methods based on exposure to infrared radiation and the chemical methods based on titration of water according to Karl Fischer.

Preferably, the dry extract of the compositions according to the invention is measured on a Mettler Toledo HG 53 balance (Halogen Moisture Analyzer).

A mascara sample (2-3 g) is deposited in an aluminium dish and subjected to a temperature of 120° C. for 60 minutes. The measurement of the dry extract corresponds to the monitoring of the mass of the sample as a function of time. The final content of solids is therefore the percentage of the final mass (at the end of 60 min) relative to the initial mass: DE=(final mass/initial mass)×100.

The composition according to the invention can be packaged in a container delimiting at least one compartment which comprises the composition, the container being closed by a closure part.

The container is preferably used in combination with an applicator, in particular in the form of a brush comprising an arrangement of hairs held by a twisted wire. Such a twisted brush is described in particular in U.S. Pat. No. 4,887,622. It can also be in the form of a comb comprising a plurality of applicational parts, obtained in particular from moulding. Such combs are described, for example, in Patent FR 2 796 529. The applicator can be integral with the container, such as described, for example, in Patent FR 2 761 959. Advantageously, the applicator is integral with a rod which, itself, is integral with the closure part.

The closure part can be coupled to the container by screwing. Alternatively, the closure part and the container can be coupled other than by screwing, in particular via a bayonet mechanism, by latching or by clamping. The term “latching” is understood to mean in particular any system which involves surmounting a flange or ring of material by elastic deformation of a portion, in particular of the closure part, and then by returning to the elastically unstressed position of the portion after the flange or ring has been surmounted.

The container can be at least partly made of thermoplastic. Mention may be made, as examples of thermoplastics, of polypropylene or polyethylene.

Alternatively, the container is made of nonthermoplastic material, in particular of glass or of metal (or alloy).

The container is preferably equipped with a wringer positioned in the vicinity of the opening of the container. Such a wringer makes it possible to wipe the applicator and optionally the rod to which it may be integrally attached. Such a wringer is described, for example, in Patent FR 2 792 618.

Preferably, the composition according to the invention is a leave-in composition.

The following examples are given by way of illustration of the present invention and cannot limit the scope thereof.

### EXAMPLES

The following compositions were produced. The amounts indicated are expressed as percentage by weight with respect to the total weight of the composition.

These mascaras are prepared according to the following procedure:

the fatty phase (wax) is heated to 98° C.,

the aqueous phase, preheated to 93° C., is added with vigorous stirring to produce the emulsion.

### Example 1a Comparative

### Mascara

| | |
|---|---|
| Beeswax | 30% |
| Sodium cocoyl glycinate (as a 30% | 16.66% |
| dispersion in water), sold under the | (i.e., 5% AM*) |
| reference Amilite GCS 12 by Ajinomoto | |
| Hydroxyethylcellulose | 0.89% |
| Simethicone (antifoaming agent) | 0.4% |
| Preservatives | q.s. |
| Water | q.s. for 100 |

AM = Active Material

### Example 1b in Accordance with the Invention

### Mascara

| | |
|---|---|
| Beeswax | 30% |
| Sodium palmamphoacetate (as a 30% | 16.66% |
| dispersion in water), sold under the | (i.e., 5% AM*) |
| reference Resassol AGP by Res Pharma, | |
| batch N3525 | |
| Hydroxyethylcellulose | 0.89% |
| Simethicone (antifoaming agent) | 0.4% |
| Preservatives | q.s. |
| Water | q.s. for 100 |

AM = Active Material

### Example 2a Comparative

### Mascara

| | |
|---|---|
| Candelilla wax | 25% |
| Sodium cocoyl glycinate (as a 30% | 16.66% |
| dispersion in water), sold under the | (i.e., 5% AM*) |
| reference Amilite GCS 12 by Ajinomoto | |
| Hydroxyethylcellulose | 0.89% |
| Black iron oxides | 5% |
| Simethicone (antifoaming agent) | 0.4% |
| Preservatives | q.s. |
| Water | q.s. for 100 |

AM = Active Material

### Example 2b in Accordance with the Invention

### Mascara

| | |
|---|---|
| Candelilla wax | 20% |
| Sodium palmamphoacetate (as a 30% | 16.66% |
| dispersion in water), sold under the | (i.e., 5% AM*) |
| reference Resassol AGP by Res Pharma, | |
| batch N3525 | |
| Hydroxyethylcellulose | 0.89% |
| Black iron oxide | 5% |
| Simethicone (antifoaming agent) | 0.4% |
| Preservatives | q.s. |
| Water | q.s. for 100 |

AM = Active Material

### Characterization

The rigidity modulus G* of each of the compositions is measured according to the protocol described above (i.e., to recapitulate, measurement in oscillation, at 25° C. (+/−1° C.), sandblasted cone/plate geometry with a diameter of 35 mm and an angle of 2°, frequency 1 Hz, stress sweep between 0.01 Pa and 1000 Pa).

The results are as follows:

| Example | G* (Pa) |
|---|---|
| 1a | 49 |
| 1b | 2500 |
| 2a | 100 |
| 2b | 620 000 |

The “texturing” nature of the surfactant according to the invention makes it possible to obtain a thicker texture for the same level of beeswax than a composition comprising an acylglycinate (composition 1a versus composition 1b).

The comparison of Example 2a with Example 2b clearly shows the advantage of the surfactant according to the invention in texturing a mascara composition: specifically, it is not necessary to employ a great deal of wax in order to obtain a creamy and thick texture (25% versus 20% respectively).

In comparison, if Example 2a is formulated with only 20% of waxes, a very fluid mixture is obtained.

### Example 3 in Accordance with the Invention

### Mascara

| Beeswax | 4.07 | |
|---|---|---|
| Carnauba wax | 3.21 | |
| Paraffin wax | 12.86 | |
| Sodium palmamphoacetate (as a 30% | 16.66 | (5% AM) |
| dispersion in water), sold under the | | |
| reference Resassol AGP by Res Pharma, | | |
| batch N3525 | | |
| Anisic acid | 1.00 | |
| Pentylene glycol | 3.00 | |
| Simethicone (antifoaming agent) | 0.13 | |
| Hydroxyethylcellulose | 0.90 | |
| Gum arabic | 3.39 | |
| Black iron oxide | 7.14 | |
| Water | q.s. for 100 | |
| SC (%) | 39.76 | (measured) |
| | 38.17 | (theoretical) |

The above written description of the invention provides a manner and process of making and using it such that any person skilled in this art is enabled to make and use the same, this enablement being provided in particular for the subject matter of the appended claims, which make up a part of the original description.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. The term “mentioned” notes exemplary embodiments, and is not limiting to certain species. As used herein the words “a” and “an” and the like carry the meaning of “one or more.”

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition comprising an aqueous phase and an emulsifying system, wherein the emulsifying system comprises at least one glycine derivative of formula (A) and/or a salt thereof:
R—CO—NH(CH₂)₂N(CH₂CH₂OH)CH₂CH₂COO⁻X⁺ (A)
where R is a carbon chain comprising at least 14 carbon atoms, and X is a cation chosen from ions of alkali metals, ions of alkaline earth metals, ammonium groups and their mixtures.

2. The composition according to claim 1, wherein the R—CO group originates from a fatty acid derived from palm oil, a fatty acid derived from olive oil, a fatty acid derived from sweet almond oil, a fatty acid derived from babassu oil, a fatty acid derived from cocoa oil, and their mixtures.

3. The composition according to claim 1, wherein X represents a cation chosen from sodium, lithium, potassium and magnesium ions, ammonium groups, and their mixtures.

4. The composition according to claim 1, wherein the glycine derivative or its salt is chosen from sodium palmamphoacetate, sodium olivamphoacetate, sodium sweetalmondamphoacetate, sodium babassuamphoacetate, sodium cacaoamphoacetate and their mixtures.

5. The composition according to claim 1, wherein the glycine derivative or its salt is present in a content ranging from 2 to 10% by weight, with respect to the total weight of the composition.

6. The composition according to claim 1, wherein the glycine derivative or its salt constitutes the main surfactant system of the composition.

7. The composition according to claim 1, wherein the glycine derivative or its salt constitutes the sole surfactant system of the composition.

8. The composition according to claim 1, wherein it comprises less than 1% by weight of triethanolamine.

9. The composition according to claim 1, wherein it is devoid of triethanolamine.

10. The composition according to claim 1, wherein it comprises less than 1% by weight of triethanolamine stearate.

11. The composition according to claim 1, wherein it is devoid of triethanolamine stearate.

12. The composition according to claim 1, wherein the aqueous phase comprises water or of a mixture of water and of at least one water-soluble solvent.

13. The composition according to claim 1, wherein the aqueous phase is present in a content of 35% to 65% by weight, with respect to the total weight of the composition.

14. The composition according to claim 1, wherein it further comprises at least one wax.

15. The composition according to claim 14, wherein the wax is present in a content of 15% to 35% by weight, with respect to the total weight of the composition.

16. The composition according to claim 1, having a dry matter content less than 45%.

17. The composition according to claim 1, wherein:
the least one glycine derivative of formula (A) and/or a salt thereof is present in the composition in 0.1 to 20% by weight with respect to the total weight of the composition,
the R—CO group originates from a fatty acid derived from palm oil, a fatty acid derived from olive oil, a fatty acid derived from sweet almond oil, a fatty acid derived from babassu oil, a fatty acid derived from cocoa oil, and their mixtures,
X represents a cation chosen from sodium, lithium, potassium and magnesium ions, ammonium groups, and their mixtures,
the least one glycine derivative of formula (A) and/or a salt thereof constitutes the main surfactant system of the composition,
the composition comprises less than 1% by weight of triethanolamine and less than 1% by weight of triethanolamine stearate,
the aqueous phase comprises water or of a mixture of water and of at least one water-soluble solvent and is present in a content of 35% to 65% by weight, with respect to the total weight of the composition, and
the composition further comprises at least one wax in a content of 15% to 35% by weight, with respect to the total weight of the composition.

18. A method for making up or for the nontherapeutic care of the eyelashes comprising the application, to the eyelashes, of the composition according to claim 1.

19. A method for making up or for the nontherapeutic care of the eyelashes comprising the application, to the eyelashes, of the composition according to claim 17.
